(19) United States
(12) Patent Application Publication
(10) Pub. No.: US 2019/0032080 A1
(43) Pub. Date: Jan. 31, 2019

(54) Recombinant Ranavirus, Methods of Production, and Its Use as a Human Vaccine Vector

(71) Applicants: James Jancovich, San Marcos, CA (US); Nathan Bartlett, Newcastle (AU)

(72) Inventors: James Jancovich, San Marcos, CA (US); Nathan Bartlett, Newcastle (AU)

(21) Appl. No.: 15/968,241

(22) Filed: May 1, 2018

Related U.S. Application Data
(60) Provisional application No. 62/500,441, filed on May 2, 2017.

(51) Int. Cl. C12N 15/86; A61K 39/12; A61P 31/16

(52) U.S. Cl. CPC C12N 15/86; A61K 2039/5254; A61P 31/16; A61K 39/12

(57) ABSTRACT

A vaccine vector comprising an attenuated, recombinant ranavirus that has at least one foreign expression element is disclosed. In other contemplated embodiments, a vaccine vector comprising a virus, wherein the virus is engineered to express at least two vaccine antigens is disclosed. In addition, methods of delivering human antigens to a mammal are disclosed that include: providing a non-mammalian virus, engineering a recombinant virus that can express at least one foreign molecule by modifying the non-mammalian virus, and using the recombinant ranavirus to deliver human antigens to a mammal.

This United States Utility patent application claims priority to U.S. Provisional Patent Application Ser. No. 62/500,441 entitled “Use of Recombinant Ranavirus as a Human Vaccine Vector” filed on May 2, 2017, which is commonly-owned and incorporated in its entirety by reference.

### FIELD OF THE SUBJECT MATTER

The field of the subject matter is the development of a vaccine that will prevent various infections.

### BACKGROUND

The annual, global cost of respiratory viral infections is in the order of billions of health care dollars. Viruses cause the common cold as well as serious lung conditions such as severe lower respiratory tract viral disease (influenza, respiratory syncytial virus (RSV)) asthma attacks (rhinovirus). School age children are the perfect vector for spread and transmission of respiratory viruses. On average children experience 5-10 colds per year, thus asthmatic kids are particularly susceptible to virus-induced asthma attacks. They are also bringing the virus home from school which can spread colds and can cause an asthma attack in susceptible family members. Indeed, in the USA there is a significant spike in hospital admissions due to asthma attacks in September, which coincides with the start of the school year after the summer break.

According to the Centers for Disease Control (CDC), “common colds are the main reason that children miss school and adults miss work. Each year in the United States, there are millions of cases of the common cold. Adults have an average of 2-3 colds per year, and children have even more. Most people get colds in the winter and spring, but it is possible to get a cold any time of the year. Symptoms usually include sore throat, runny nose, coughing, sneezing, watery eyes, headaches and body aches. Most people recover within about 7-10 days. However, people with weakened immune systems, asthma, or respiratory conditions may develop serious illness, such as pneumonia. The CDC also links rhinovirus infections to sinus and ear infections. In addition, RV infections are highly linked to the development of asthma as well as exacerbate disease in chronic obstruction pulmonary disorder and cystic fibrosis which predisposes individuals to secondary bacterial infections and pneumonia, which can be life threatening. Lung transplant patients are also at risk from respiratory viral infections, also due to secondary bacterial pneumonia. Taken together (100s of subtypes, frequency of infection, ease of transmission by susceptible school-age children, lack of vaccine), it is little wonder that RV are the most common trigger of asthma attacks and infections that can—at the least, impact productivity and at worst—be life-threatening. Prevention of RV infections has real potential to impact on the huge health care burden directly attributable to this virus. Therefore, it would be ideal to find a vaccine that would help combat at least the rhinovirus-associated infections.

Other respiratory viruses such as influenza and RSV also continue to cause colds and more severe respiratory diseases in the very young and the elderly. There is no global vaccine for influenza and the viral subtypes that constitute the vaccine need to be updated annually. The effectiveness of the influenza vaccine varies considerably from year to year and may be as low as 10% protective (as was the case in 2017 for seasonal H3N2). For RSV (like RV) there is no effective vaccine.

We have incredibly successful vaccines for most important childhood viral diseases, but surprisingly not RV, influenza or RSV. The main reason is that these viruses constitute hundreds of genetically distinct subtypes that all require a specific protective immune response. Any vaccine would need to generate protection against all of these circulating subtypes. In practice, it is likely that there will be clusters of cross subtype-protection whereby a vaccine will need cover a lower number of representative viruses. Nonetheless, the number of antigens needed for a vaccine will still be substantial. Thus, any candidate vaccine vector is going to need a large vaccine antigen expression capacity. In this respect, it may mean that new and unique research opportunities have to be reviewed to find a solution to the mosaic cluster of respiratory viruses.

### BRIEF DESCRIPTION OF THE FIGURES

FIG. 1 shows the results of the mouse safety trial.

FIG. 2 shows the results of the antibody response in infected mice.

FIG. 3 shows a schematic of process for generating a recombinant ranavirus.

FIG. 4 shows GFP expression from a recombinant ATV.

FIG. 5A shows expression of GNR construct from recombinant ATV in mouse lung epithelial cells. Mouse lung epithelial (LA4) cells were either mock infected or infected with either wild-type ATV or ATVΔ40L that expresses the GNR construct at a multiplicity of infection of 1 or 10 at 31° C.

FIG. 5B shows expression of GNR construct from recombinant ATV in mouse lung epithelial cells. Mouse lung epithelial (LA4) cells were either mock infected or infected with either wild-type ATV or ATVΔ40L that expresses the GNR construct at a multiplicity of infection of 1 or 10 at 35° C.

FIG. 5C shows expression of GNR construct from recombinant ATV in mouse lung epithelial cells at 31° C.

### SUMMARY OF THE SUBJECT MATTER

A vaccine vector comprising an attenuated, recombinant ranavirus that has at least one foreign expression element is disclosed. In other contemplated embodiments, a vaccine vector comprising a virus, wherein the virus is engineered to express at least two vaccine antigens is disclosed.

In addition, methods of delivering human antigens to a mammal are disclosed that include: providing a non-mammalian virus, engineering a recombinant virus that can express at least one foreign molecule by modifying the non-mammalian virus, and using the recombinant ranavirus to deliver human antigens to a mammal.

In contemplated embodiments, engineering a recombinant virus includes: generating a recombination cassette, wherein the cassette contains homologous sequences flanking a screenable and selectable reporter gene driven by a promoter, infecting at least one cell with the attenuated non-mammalian virus, transfecting the at least one cell with the recombination cassette to form a combination of the at least one cell and the non-mammalian virus, harvesting a modified combination of the at least one cell and the attenuated non-mammalian virus; and selecting from the modified combination the recombinant virus deleted of the target open reading frame or ORF by serial passaging in cells treated with selection specific components.

### DETAILED DESCRIPTION

In view of earlier-presented information, the ideal vector for a human vaccine is a large DNA virus that can be engineered to express multiple vaccine antigens. Importantly, this virus should not productively infect human cells. Instead, it needs to enter human cells, produce the vaccine antigens but not form a new virus, which is called abortive replication. The best place to find such a viral vaccine vector is to look in animals that are very distantly related to humans.

Specifically, a contemplated vaccine vector comprises an attenuated, recombinant ranavirus that has at least one foreign expression element. Contemplated recombinant, attenuated viruses are unique in that they have been deleted of pathogenesis genes and those genes are replaced with expression constructs, for example and including mammalian promoter elements driving expression of at least one antigen.

As used herein, the term “attenuated” with respect to a virus or vaccine vector means a vaccine created by reducing the virulence of a pathogen, but still keeping it viable (or “live”). Attenuation takes an infectious agent and alters it so that it becomes harmless or less virulent. These vaccines are in contrast to those produced by “killing” the virus (inactivated vaccine). An attenuated virus may be used to make a vaccine that is capable of stimulating an immune response and creating immunity in a patient, but not of causing illness in that same patient. In contemplated embodiments, viruses have been deleted of pathogenesis genes. There are currently 4 loci/genes in the contemplated virus that can be deleted and foreign material inserted; however, in other contemplated embodiments, other loci or genes or numbers of loci or genes can be deleted and foreign material inserted. In some contemplated embodiments, in place of the pathogenesis gene(s), a mammalian virus promoter element and a human translation enhancement element have been inserted that drive expression of a foreign antigen.

In contemplated embodiments, the at least one foreign expression element expresses at least one foreign protein, at least two vaccine antigens, at least one virus-like particle or a combination thereof.

In some contemplated embodiments, a vaccine vector comprises a virus, wherein the virus is engineered to express at least two vaccine antigens. In some of these contemplated embodiments, the virus is an attenuated recombinant ranavirus.

In addition, methods of delivering human antigens to a mammal are disclosed that include: providing a non-mammalian virus, engineering a recombinant virus that can express at least one foreign molecule by modifying the non-mammalian virus, and using the recombinant ranavirus to deliver human antigens to a mammal.

In contemplated embodiments, engineering a recombinant virus includes: generating a recombination cassette, wherein the cassette contains homologous sequences flanking a screenable and selectable reporter gene driven by a promoter, infecting at least one cell with the attenuated non-mammalian virus, transfecting the at least one cell with the recombination cassette to form a combination of the at least one cell and the wild-type non-mammalian virus, harvesting a modified combination of the at least one cell and the attenuated non-mammalian virus; and selecting from the modified combination the recombinant virus deleted of the target open reading frame or ORF by serial passaging in cells treated with selection specific components.

As disclosed herein, contemplated vaccine vectors can be used to reduce the occurrence of mammalian respiratory disease and/or related diseases or conditions.

All animals, including cold-blooded amphibians, are host to viruses, including salamanders. Salamander models have been used in other research related to human conditions. For example, Del Priore et al. looked at salamander research to find a connection between retinal cell apoptosis and increasing age. (Lucian V. Del Priore, Ya-Hui Kuo and Tongalp H. Tezel, “Age-Related Changes in Human RPE Cell Density and Apoptosis Proportion In Situ”, Investigative Ophthalmology & Visual Science, October 2002, Vol. 43, 3312-3318 citing Townes-Anderson E, Colantonio A, St Jules RS. “Age-related Changes in the Tiger Salamander Retina”, Exp Eye Res. 1998; 66:653-667) Wagner et al. used fish models, including aquatic salamanders to show that there is evidence of a stanniocalcin-like hormone in humans, specifically human kidneys. (Graham R. Wagern, Collete C. Guiraudon, Christine Milliken and D. Harold Copp, “Immunological and Biological Evidence for a Stanniocalcin-like Hormone in Human Kidney”, Proc. Natl. Acad. Sci. USA, 92 (1995).

As a basis for this research, Arizona salamanders were captured and, upon investigation, showed signs of illness. After significant examination and analysis, a new virus, now called Ambystoma tigrinum virus (ATV), was found. This virus is a member of the family Iridoviridae—the members of which are large DNA viruses that infect insects, amphibians, reptiles and fish.

Since this discovery, the researchers spent several years perfecting the technique for making recombinant ATVs (recATV) and other ranaviruses that express foreign proteins (recRanaV). For example, a recRanaV was created that expresses a protein (green fluorescent protein—GFP) that causes infected cells to glow green. In addition, a mouse model system was developed for RV infections and test compounds, and other agents to fight disease, are routinely tested in this model system.

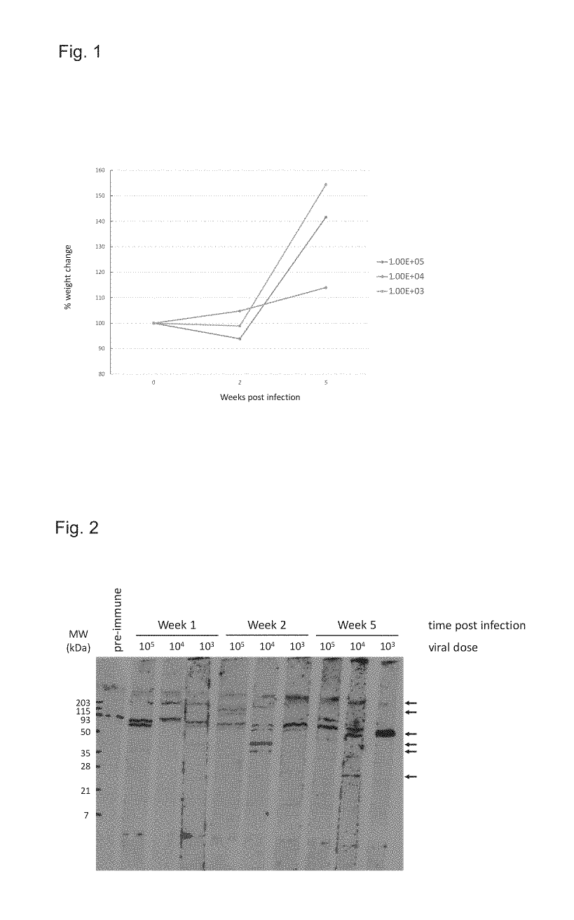

The new recRanaV will be utilized, as disclosed herein, in mouse studies to prove that it can function as a vaccine vector. The recRanaV vector will be used as a human vaccine vector to deliver protective RV antigens. FIGS. 1-5 show some of the preliminary results and information related to this invention.

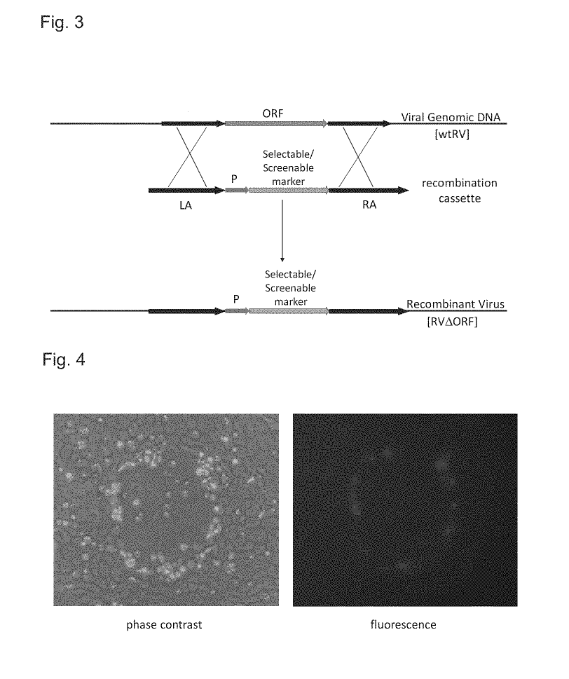

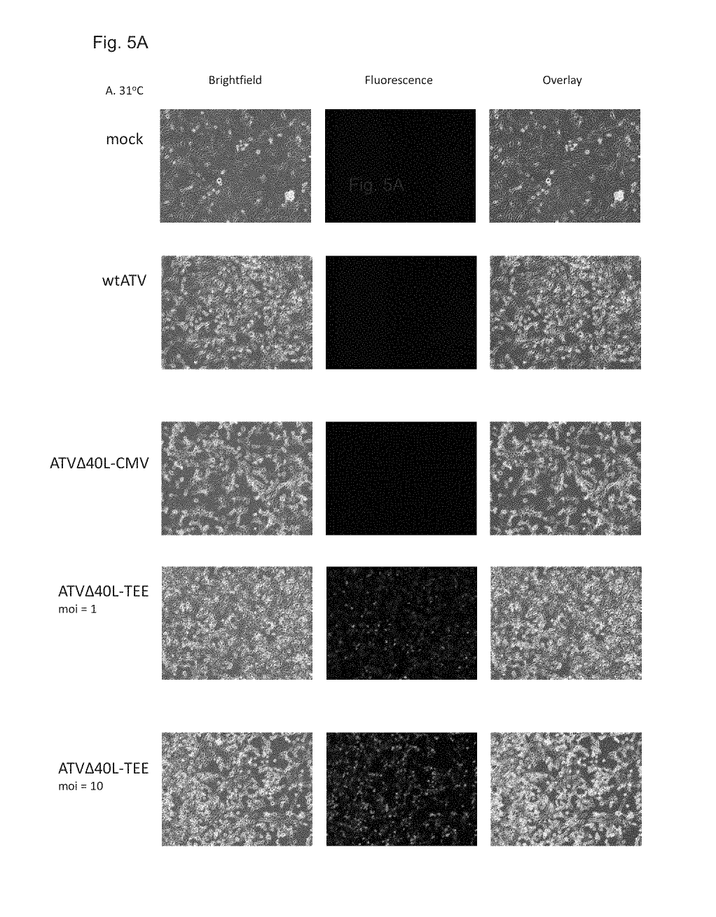

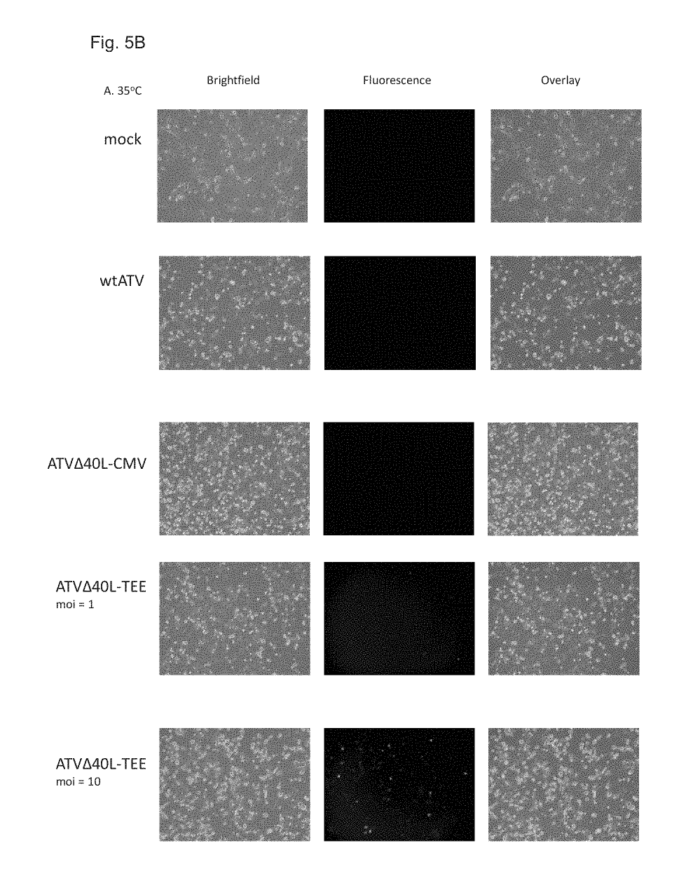

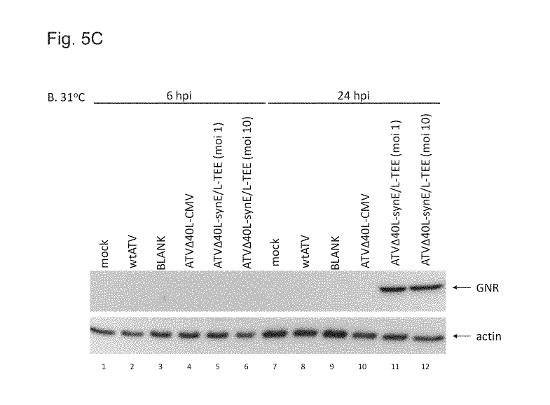

Specifically, the data show that ATV intranasal infections of mice is non-pathogenic as all mice infected with virus did not show significant weight loss throughout the experiment or display any signs or symptoms of disease (FIG. 1). In addition, mice infected with ATV produced an immune response specific to the virus (FIG. 2). These data suggest that ATV is a safe virus that produces antibodies to specific viral proteins. However, these experiments were performed with wild-type ATV that does not express a foreign antigen. Therefore, the ability to generate a mutant ATV expressing green fluorescent protein (GFP) that is fused to a selectable marker, neomycin resistance (FIG. 3) was developed. This construct, termed GNR for the GFP and neomycin resistance gene used to make the virus, is easily expressed in fish cells that are susceptible and permissive to ATV (FIG. 4). Since the vaccine contemplated herein is for mammalian respiratory disease, it has been shown that expression of the GNR construct in mouse lung epithelial cells (FIG. 5). Expression of GNR from ATV is temperature sensitive with reduced expression at 35° C. as compared to 31° C. and no expression was observed at 37° C. Collectively, the data suggest a novel antigen delivery system that can be used to develop vaccines for mammalian (i.e. human) respiratory disease has been developed. Each of these figures will be described in detail below.

FIG. 1 shows the results of the mouse safety trial. Mice were infected intranasally with 100 μl of wild-type ATV (wtATV) at 10⁵, 10⁴ and 10³ viral particles or mock infected with medium alone. The health of the mock or virus infected mice was assessed by monitoring their weight over a 5-week period. Preliminary data represent one mouse/dose/time point. Mice were infected with a single dose of virus (i.e. no boost).

FIG. 2 shows the results of the antibody response in infected mice. Blood was taken by heart puncture from infected mice at day 0 (pre-immune) and weeks 1, 2 and 5 post infection. Serum was isolated and used to detect ATV specific proteins by Western blot. The pre-immune serum is the control, so bands in the blot that are not present in the pre-immune serum lane indicate virus specific proteins were recognized by the host's immune system. Mice were assayed for the production of IgM (A) and IgG (B) specific immune responses to the virus.

FIG. 3 shows a schematic of process for generating a recombinant ranavirus. The process of generating a knock-out ranavirus (RV) deleted of the target gene requires the generation of a recombination cassette that contains homologous sequences (LA and RA) flanking a screenable and selectable reporter gene driven by a promoter (P). Cells are infected with wild-type virus and then transfected with the recombination cassette. Cells and virus are harvested after 48 hours and the recombinant virus deleted of the target ORF is selected by serial passaging in cells treated with selection specific components. Recombinant virus deleted of the target ORF will be resistant to the selection substance and produce easily observable plaques.

FIG. 4 shows GFP expression from a recombinant ATV. ATV mutant virus plaque under phase contrast and fluorescent microscopy.

FIGS. 5A-C shows expression of GNR construct from recombinant ATV in mouse lung epithelial cells. Mouse lung epithelial (LA4) cells were either mock infected or infected with either wild-type ATV or ATVΔ40L that expresses the GNR construct at a multiplicity of infection of 1 or 10 at 31° C., 35° C. or 37° C. Cells were: (A) analyzed by florescent microscopy for GFP expression; or (B) harvested at the indicated time points and total proteins were isolated before analysis for GNR expression by Western blot. Data for the 37° C. are not shown as not GFP expression was not observed at this temperature. FIG. 5A shows expression of GNR construct from recombinant ATV in mouse lung epithelial cells. Mouse lung epithelial (LA4) cells were either mock infected or infected with either wild-type ATV or ATVΔ40L that expresses the GNR construct at a multiplicity of infection of 1 or 10 at 31° C. FIG. 5B shows expression of GNR construct from recombinant ATV in mouse lung epithelial cells. Mouse lung epithelial (LA4) cells were either mock infected or infected with either wild-type ATV or ATVΔ40L that expresses the GNR construct at a multiplicity of infection of 1 or 10 at 35° C. FIG. 5C shows expression of GNR construct from recombinant ATV in mouse lung epithelial cells at 31° C.

### Materials and Methods

The following materials and methods were used to obtain and collect the data presented herein.

Cells and Virus

Fathead minnow (FHM; ATCC CCL-42) cells were maintained in Minimum Essential Medium with Hank Salts (HMEM) (Gibco) supplemented with 5% fetal bovine serum (FBS) (Hyclone) and 0.1 mM nonessential amino acids and vitamins (Invitrogen). FHM cells were incubated at 20 to 22° C. in the presence of 5% CO₂. LA4 mouse lung epithelial cells (kindly provided by Dr. Bianca Mothé and the La Jolla Institute of Allergy and Immunology) were maintained in F12K medium supplemented with 15% FBS and incubated at 37° C. with 5% CO₂. Wild-type Ambystoma tigrinum virus (wtATV), was originally isolated from tiger salamanders in Southern Arizona (Jancovich et al., 1997). Wild-type and mutant ATV were amplified and quantified in FHM cells. Briefly, viral amplification was performed in 100 mm dishes of FHM cells that were infected with virus at a multiplicity of infection of 0.01, rocked for 1 hr and then overlayed with HMEM with 5% FBS. Infected cells were monitored for cytopathic effects (CPE). Once CPE reached 95-100%, infected cells were harvested, concentrated by centrifugation at 1,000×g for 10 min and the pellet of infected cells resuspended in 100 μl of 10 mM Tris, pH 8.0. Virus was released by 3 cycles of freeze/thaw followed by centrifugation at 1,000×g for 10 min to clarify cellular debris. The supernatant containing virus was quantified by plaque assay in FHM cells.

Generating Recombination Cassettes

Recombination cassettes to delete a target gene, or open reading frame (ORF) and insert a foreign antigen were generated by designing forward (for) and reverse (rev) primers to amplify the upstream (LA) and downstream (RA) flanking sequences of the gene to be deleted. Primers were designed to initially amplify a PCR product around 1,000 nt up- and downstream from the start and end of the target sequence, respectively. These primers (ORF#_LA_for_1k and ORF#_RA_rev_1k, respectively) were paired with primers designed immediately before the start (ORF#_LA_rev) and after the end (ORF#_RA_rev) of the target gene. An adapter sequence (AF; 5′ GGTATAGGCGGAAGCGCC 3′) was added to the 3′ end of the LA reverse primer (AF_ORF#_LA_rev) and a second adapter (AR; 5′ GAACAGAAACTGATTAGCGAAGAAGAC 3′) was added to the 5′ end of the RA forward primer (AR_ORF#_RA_for). Each of these primers were designed to have a predicted melting temperature around 60° C. Pairing the ORF#_LA_for_1k primer with the AF_ORF#_LA_rev and the AR_ORF#_RA_for with ORF#_RA_1k_rev generated approximately 1 kb of sequence of both the left and right flanking homologous sequences with adapters at the 3′ end of the LA and the 5′ end of the RA. Using primers AF-p for and AR-NeoR rev, which target the promoter (p)-green fluorescent protein (GFP)-neomycin resistance gene, which we will refer to as pGNR, was PCR amplified using a pcDNA3.1 vector containing the GNR construct as a template. For each PCR reaction, 50 ng of plasmid or 100 ng of viral DNA was added to the High Fidelity PCR Master Mix according to the manufacturer's instructions (Roche) and DNA was amplified with a single cycle of 94° C. for 2 minutes, followed by 25 cycles of 94° C. (30 seconds), 50° C. (for primer sets seq for/rev and 500_for/rev) or 55° C. (for primer set 1k_for/rev) (30 seconds), 72° C. (90 seconds) and a final cycle of 72° C. for 7 minutes. PCR products were visualized by 1% agarose gel electrophoresis and products were purified by Wizard® SV Gel and PCR Clean-Up System (Promega) system as described by the manufacturer after excision from 0.7% agarose gel. Purified PCR products were quantified by Nanodrop spectrophotometry. At this point we have three purified PCR products for each ATV ORF: the LA, RA and pGNR.

To generate a recombination cassette by overlapping PCR, 50 ng of each PCR product (LA, RA and pGNR) was added to 45 μl reaction (final volume) containing 1× iProof HF buffer, 200 μM of each dNTP, and 0.02 U/μl iProof DNA polymerase (BioRad). The recombination cassette assembly was initiated by a single cycle of 98° C. (30 seconds), followed by 7 cycles of 98° C. (10 seconds), 58° C. (28 minutes), 72° C. (150 seconds). After the completion of this program, 0.5 μM of the ORF#_LA_1k_for and ORF#_RA_1k_rev were added along with another 0.02 U/μl iProof DNA polymerase. The reaction was then returned to the thermocycler and a second program consisting of a single cycle of 98° C. (30 seconds), followed by 35 cycles of 98° C. (10 seconds), 55° C. (30 seconds), 72° C. (150 seconds) and a final cycle of 72° C. for 5 minutes was performed. PCR products were visualized and purified as described above. Purified recombination cassettes were then re-amplified using the ORF#_LA_500_for and ORF#_RA_500_rev primers using the High Fidelity PCR Master Mix as described above. PCR products were visualized and purified as described above and then cloned into pCR2.1®-TOPO® cloning vector as per the manufacturer's instructions (Thermo Fisher Scientific). Colonies were screened for the recombination cassette using the seq for/rev primer set for each ORF and correctly constructed recombination cassettes were confirmed by sequencing. The recombination cassette was PCR amplified from the plasmid, agarose gel purified and quantified as described above for use in generating a knockout virus.

Generating Knockout ATV

Approximately 50% confluent monolayers of FHM cells in 35 mm dishes were infected with wtATV at a MOI of 0.01 for 1 hour at room temperature. While the virus was attaching, 500 ng of the target ATV ORF recombination cassette that had been PCR amplified and purified was added to FuGene® 6 transfection reagent according to the manufacturer's instructions (Promega). This solution was incubated at room temperature for 20 minutes. After 1 hour, the virus inoculum was removed and replaced with the DNA-FuGene® 6 mixture. Cells were rocked with the transfection mixture for 1 hour at room temperature. After rocking, the infected/transfected cells were overlayed with 1×HMEM medium containing 5% FBS and incubated for 48 hours. Infections were then harvested and subjected to three rounds of freeze-thaw to release virus from the cell. The sample was then clarified by centrifugation at 1,000×g for 10 minutes and recombinant viruses were selected by multiple blind passages in confluent monolayers of FHM cells in the presence of 1 mg/mL G418 (i.e. neomycin). wtATV, which is sensitive to G418, was used as a control. The presence of a GFP expressing, neomycin resistant virus plaque was indicative of the generation of a recombinant ATV with a knock-out of the target gene. GFP-neomycin resistant virus was then plaque purified up to four times in the presence of 1 mg/ml G418, grown to high titers as described above and viral DNA was isolated as previously described (Jancovich and Jacobs, 2011). PCR confirmation of the ORF knock-out virus and sequencing around the ATV gene of interest was performed using the seq for/rev primer pair described above.

RT-PCR Analysis of Cellular Gene Expression

Total RNA from infected cells was extracted using Qiashredder columns followed by RNA isolation using the RNeasy kit as described by the manufacturer (Qiagen). RNA was quantified by spectrophotometric analysis and cDNA was synthesized from 1 μg of total RNA using random primers and the SuperScript® III Reverse Transcriptase (Invitrogen Life Technologies) as directed by the manufacture. Amplification of specific genes, including GNR, was performed. PCR reactions (50 μl) were performed using the High Fidelity Taq Polymerase Master Mix kit (Roche Diagnostics). Reactions were incubated at 94° C. for 2 minutes followed by 25 cycles of 94° C. for 30 seconds, 55° C. for 30 seconds, and 72° C. for 90 seconds, and a final elongations cycle of 72° C. for 7 minutes. Amplified products were separated on a 1% agrose gel electrophoresis and visualized using a G:Box imaging platform (Syngene).

Cell Extractions and Western Blot Analysis

Infected cell lysates were collected in 1×SDS sample buffer (50 mM Tris, pH 6.8; 2% SDS; 0.1% bromophenol blue; 10% glycerol; 100 mM betamercaptoethanol) before purification by Qiashredder collection column (Qiagen). Equal cell volumes of cellular extracts were subjected to SDS-PAGE on 12% polyacrylamide gels. Proteins were transferred to either a nitrocellulose membrane or a PVDF membrane at 100 volts for 60 minutes in 10 mM CAPS, pH 11.0, with 20% methanol and 14 mM 2-mercaptoethanal. The blot was blocked for 1 hour in 1×TBS with milk (20 mM Tris-HCl [pH 7.8]; 180 mM NaCl; 3% nonfat dry milk). The blots were incubated overnight at 4° C. with primary antibodies at the appropriate dilution as outlined by the manufacturer (Abcam). Primary antibodies were removed, and the blot was washed three times with 1×TBS containing milk for 30 minutes at room temperature. The blot was then probed with a 1:15,000 dilution of goat anti-rabbit or rabbit anti-mouse IgG-peroxidase conjugate antibody (Sigma) for 1 hour at room temperature. These secondary antibodies were then removed, and the blot was washed three times for 10 minutes each in 1×TBS with milk and then washed three times for 5 minutes each in 1×TBS without milk. The blot was visualized after treatment with the Super Signal Dura chemiluminescent kit according to the manufacturer's instructions using the G:Box imaging platform (Syngene). The relative intensity of proteins was quantified using the GeneTools analysis software (Syngene).

Mouse Safety Trial

BALB/c mice were infected intranasally with 100 μl of wtATV at 10⁵, 10⁴ and 10³ viral particles. The health of the virus infected mice was assessed by monitoring their weight over a 5 week period. Mice were infected with a single dose of virus (i.e. no boost) and blood was harvested by cardiac puncture at 2 and 5 weeks post infection. Blood serum was isolated and used to assess antibody production in mice infected with virus. To detect a virus specific immune response, FHM cells were infected with wtATV at a MOI of 5 and virus and cells were harvested and proteins were isolated at 12 hours post infection. Proteins in the infected cell extracts were separated by SDS-PAGE before transfer to nitrocellulose. Western blots were performed as described above using mouse specific anti-IgM antibodies to detect a virus-induced immune response in the infected mouse.

Thus, specific embodiments of a recombinant ranavirus, along with methods of use of contemplated recombinant ranavirus as a human vaccine vector have been disclosed. It should be apparent, however, to those skilled in the art that many more modifications besides those already described are possible without departing from the inventive concepts herein. The inventive subject matter, therefore, is not to be restricted except in the spirit of the disclosure herein. Moreover, in interpreting the specification, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

1. A vaccine vector comprising an attenuated, recombinant ranavirus that has at least one foreign expression element.

2. The vaccine vector of claim 1, wherein the expression element expresses at least one foreign protein.

3. The vaccine vector of claim 1, wherein the expression element expresses at least two vaccine antigens.

4. The vaccine vector of claim 1, wherein the expression element expresses at least one virus-like particle.

5. A vaccine vector comprising a virus, wherein the virus is engineered to express at least two vaccine antigens.

6. The vaccine vector of claim 5, wherein the virus is an attenuated recombinant ranavirus.

7. A use of the vaccine vector of claim 1 to reduce the occurrence of mammalian respiratory disease.

8. A use of the vaccine vector of claim 5 to reduce the occurrence of mammalian respiratory disease.

9. A method of delivering human antigens to a mammal, comprising:
providing a non-mammalian virus,
engineering a recombinant virus that can express at least one foreign molecule by modifying the non-mammalian virus, and
using the recombinant ranavirus to deliver human antigens to a mammal.

10. The method of claim 9, wherein the non-mammalian virus comprises an amphibian virus.

11. The method of claim 10, wherein the amphibian virus comprises Ambystoma tigrinum virus.

12. The method of claim 9, wherein the at least one foreign molecule comprises at least one foreign protein, at least two vaccine antigens, at least one virus-like particle, or a combination thereof.

13. The method of claim 9, wherein engineering a recombinant virus comprises:
generating a recombination cassette, wherein the cassette contains homologous sequences flanking a screenable and selectable reporter gene driven by a promoter,
infecting at least one cell with the attenuated non-mammalian virus,
transfecting the at least one cell with the recombination cassette to form a combination of the at least one cell and the non-mammalian virus,
harvesting a modified combination of the at least one cell and the attenuated non-mammalian virus, and
selecting from the modified combination the recombinant virus deleted of the target open reading frame or ORF by serial passaging in cells treated with selection specific components.

14. The use of the method of claim 9 to reduce the occurrence of mammalian respiratory disease.

15. The use of the method of claim 13 to reduce the occurrence of mammalian respiratory disease.
